(19) United States
(12) Patent Application Publication
(10) Pub. No.: US 2025/0194944 A1
(43) Pub. Date: Jun. 19, 2025

(54) STRESS PERFORMANCE TRAINING SYSTEM

(71) Applicant: Neurosmart Inc., Mountain View, CA (US)

(72) Inventors: Melis Yilmaz Balban, Mountain View, CA (US); Yang Liu, Pasadena, CA (US)

(21) Appl. No.: 18/538,785

(22) Filed: Dec. 13, 2023

(51) Int. Cl. A61B 5/0533; A61B 5/024; A61B 5/16

(52) U.S. Cl. CPC A61B 5/0533; A61B 5/02416; A61B 5/165

(57) ABSTRACT

A stress performance training system may monitor sensor data that describes in part physiological data of a user collected over a time period. The sensor data includes galvanic skin response (GSR) data for the user. The system may determine a target period within the time period. The target period corresponds to an occurrence of the user experiencing a stressor. The system may pre-preprocess, for at least some of the time period including the target period, the GSR data to determine one or more performance scores of the user. The system may present the one or more performance scores for stress management. In some embodiments, the system may alert the user if a performance score of the one or more performance scores does not satisfy a threshold value. In some embodiments, the alert may include a recommended course of action for improving the performance score.

### FIELD OF THE INVENTION

This disclosure relates generally to training systems, and more specifically to stress performance training systems.

### BACKGROUND

Practitioners of some professions are subject to stressful (and often high stakes) conditions. For example, police officers, fireman, elite athletes, etc. in the course of their duties are often required to quickly make high stakes decisions. While there are conventional devices that provide gross monitoring of physiological data (e.g., heart rate) of users, such systems often simply monitor user physiology without linking the monitored physiological data to a relevant performance indicator for a high stress event. As such, a technical solution is missing for objectively training and evaluating user performance during stressful conditions.

### SUMMARY

In accordance with one or more aspects of the disclosure, a stress performance training system is described. The system may include, e.g., one or more sensor assemblies, one or more client devices, a trainer device, and a stress management system. A user is associated with a client device and a sensor assembly. The sensor assembly collects sensor data that describes in part physiological data (e.g., galvanic skin response (GSR) data) of the user while the user experiences a stressor and provides it to the client device. In some embodiments, the client device may process the sensor data to determine one or more performance scores for stress management including a stress regulation score. For example, the client device may determine a target period within a time period of the stressor, the target period corresponding to an occurrence of the user experiencing a stressor. The client device may pre-process, for at least some of the time period including the target period, the GSR data to generate a tonic component associated with the GSR data, a phasic component associated with the GSR data, or both. The client device may generate a representation of the tonic component associated with the GSR data. In one such example, the client device generates a histogram using the tonic component associated with the GSR data. In other examples, the client device may generate a best fit line representing the tonic component of the data, or may generate a different representation of the data that characterizes the tonic component of the GSR data. The client device may determine a stress regulation score using the histogram or other representation. The stress regulation score describes a capability of the user to regulate stress during the target period. In some embodiments, the client device may present, e.g., the one or more performance scores including the stress regulation score to the trainee. The client device may provide, via a network, stress performance data (e.g., sensor data, the one or more performance scores, etc.) to the trainer device and/or the stress management system. In some embodiments, the client device, the trainer device, the stress management system may perform an action (e.g., provide an alert to the trainee) based in part on the one or more performance scores.

The trainer device may be used (e.g., by a trainer) to evaluate performance of one or more users (e.g., trainees) during stressor events. The trainer device may receive (e.g., via a network) stress performance data from one or more client devices that are each associated with different users. The trainer device may analyze the stress performance data to generate performance information. The trainer device may present the performance information including one or more performance scores for the one or more trainees. In some embodiments, the trainer device may determine that a value of a performance score of a user is below a threshold value. The trainer device may generate an instruction to provide an alert to a client device associated with the user, and provide the alert to the client device. Responsive to receiving the alert, the client device may present the alert. In some embodiments, the alert may include a course of action (e.g., disengage and perform breathing exercises) for the user to take to help address the deficient performance score.

In some embodiments, the stress management system may perform an action (e.g., send a client device an alert) based on received stress performance data. The stress management system may apply the stress performance data to a machine learned model to generate one or more visualizations. The stress management system may perform an action (e.g., provide feedback to the user) based in part on the one or more visualizations.

In some aspects, the techniques described herein relate to a method including: receiving sensor data that describes in part physiological data of a user collected over a time period, the sensor data including GSR data for the user; determining a target period within the time period, the target period corresponding to an occurrence of the user experiencing a stressor; pre-processing, for at least some of the time period including the target period, the GSR data to determine a tonic component associated with the GSR data; generating a histogram (or other representation) using the tonic component of the GSR data; determining a stress regulation score using the histogram (or other representation), the stress regulation score describing a capability of the user to regulate stress during the target period; and presenting, via a display, one or more performance scores including the stress regulation score.

In some aspects, the techniques described herein relate to a non-transitory computer-readable storage medium including stored instructions, the instructions when executed by a processor of a device, cause the device to: receive sensor data that describes in part physiological data of a user collected over a time period, the sensor data including GSR data for the user; determine a target period within the time period, the target period corresponding to an occurrence of the user experiencing a stressor; pre-process, for at least some of the time period including the target period, the GSR data to determine a tonic component associated with the GSR data; generate a histogram (or other representation) using the tonic component of the GSR data; determine a stress regulation score using the histogram (or other representation), the stress regulation score describing a capability of the user to regulate stress during the target period; and present, via a display, one or more performance scores including the stress regulation score.

In some aspects, the techniques described herein relate to a system including: a sensor assembly configured to collect sensor data that describes in part physiological data of a user over a time period, the sensor data including GSR data for the user; and a client device communicatively coupled to the sensor assembly, the client device including: a display, and a controller configured to: determine a target period within the time period, the target period corresponding to an occurrence of the user experiencing a stressor, pre-process, for at least some of the time period including the target period, the GSR data to determine a tonic component associated with the GSR data, generate a histogram (or other representation) using the tonic component of the GSR data, determine a stress regulation score using the histogram (or other representation), the stress regulation score describing a capability of the user to regulate stress during the target period, and instruct the display to present one or more performance scores including the stress regulation score.

### BRIEF DESCRIPTION OF THE DRAWINGS

FIG. 1 illustrates an example environment for a stress performance training system, in accordance with one or more embodiments.

FIG. 2 illustrates a block diagram of a client device, in accordance with one or more embodiments.

FIG. 3 is an example stress performance graphical user interface, in accordance with one or more embodiments.

FIG. 4A is a chart showing skin response and an associated histogram for a user who regulates stress well during a target period, in accordance with one or more embodiments.

FIG. 4B is a chart showing skin response and an associated histogram for a user who does not regulate stress well during a target period, in accordance with one or more embodiments.

FIG. 5 is a flowchart illustrating a process for evaluating stress performance on a client device, in accordance with one or more embodiments.

FIG. 6 is a flowchart illustrating a process for evaluating stress performance of multiple users on a trainer device, in accordance with one or more embodiments.

FIG. 7 is a flowchart illustrating a process for evaluating stress performance of multiple users using a machine learned model, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

In accordance with one or more aspects of the disclosure, a stress performance training system is described. A stressor is an experience whose participants are subject to large amounts of stress. A stressor may be, e.g., participating in a hostage negotiation, providing medical treatment to someone who is grievously injured, clearing a building of hostile terrorists, etc. Note that stressors can occur in a variety of contexts. For example, in the context of sports, a stressor could be having to score in order to win a game (e.g., 1 second left on the clock and having to make the field goal for your team to win). The stress performance training system monitors physiological data of a user, and evaluates how well the user performs during a stressor based in part on the physiological data. In some embodiments, the stress performance training system may be used to evaluate how a user performed during a stressor and provide recommended courses of action to improve future performance. In some embodiments, the stress performance training system may provide feedback to the user in real time regarding their performance in dealing with a stressor. In this manner, the stress performance training system can provide a means to improve how well a user performs in stressful situations.

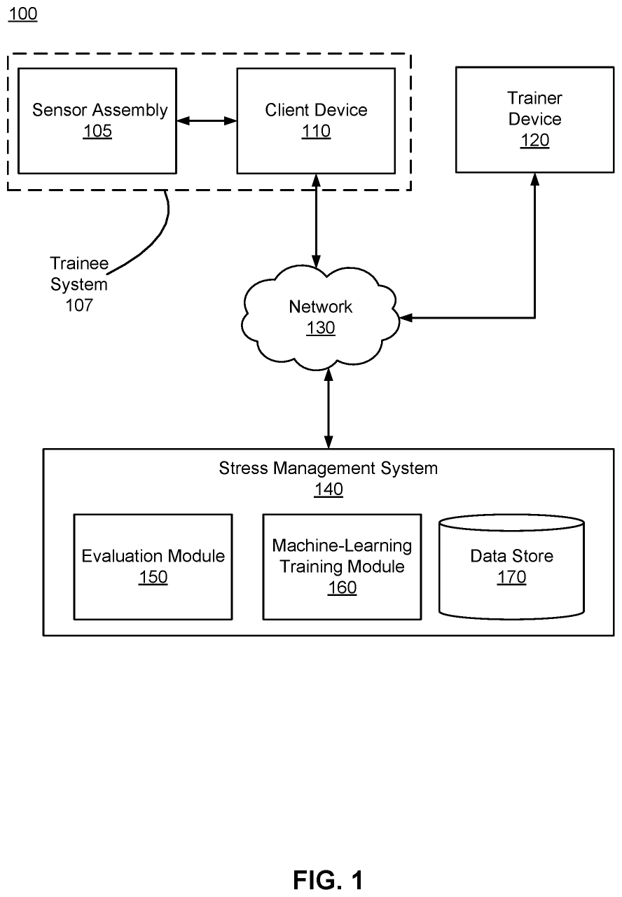

FIG. 1 illustrates an example environment for a stress performance training system 100, in accordance with one or more embodiments. The environment illustrated in FIG. 1 includes a sensor assembly 105, a client device 110, a trainer device 120, a network 130, and a stress management system 140. The sensor assembly 105 of a user and the client device 110 of the user together form a trainee system 107 for that user. Alternative embodiments may include more, fewer, or different components from those illustrated in FIG. 1, and the functionality of each component may be divided between the components differently from the description below. For example, the client device 110 and/or the trainer device 120 may have some or all of the functionality of the stress management system 140. Additionally, each component may perform their respective functionalities in response to a request from a human, or automatically without human intervention.

The sensor assembly 105 collects sensor data that describes in part physiological data of a user over a period of time. The sensor assembly 105 may be part of a wearable device that includes a plurality of sensors. For example, the wearable device may be a watch and/or wristband. The sensor assembly 105 collects the sensor data using the plurality of sensors. The plurality of sensors includes skin conductivity sensors that collect galvanic skin response (GSR) data of the user. The sensor assembly 105 may also include, at least one position sensor, at least one photoplethysmogram (PPG) sensor, some other sensor configured to monitor physiological data of the user, or some combination thereof. A position sensor generates position data in response to motion of the position sensor. The position sensor may include an inertial measurement unit (IMU). Examples of position sensor include: one or more accelerometers, one or more gyroscopes, one or more magnetometers, another suitable type of sensor that detects motion, a type of sensor used for error correction of the IMU, or some combination thereof. The position sensor may be located external to the IMU, internal to the IMU, or some combination thereof. A PPG sensor includes one or more light sources and one or more light detectors. The PPG sensor may be configured to monitor, e.g., heart rate of the user, blood oxygenation of the user, blood pressure, etc., and output corresponding PPG data. The sensor assembly 105 is communicatively coupled (e.g., wired and/or wireless connection) to the client device 110. For example, sensor assembly 105 and the client device 110 may include Bluetooth or near-field communication (NFC) technologies or protocols for local communications. The sensor assembly 105 may transmit encrypted or unencrypted sensor data.

The client device 110 is a device through which a user may interact with the sensor assembly 105, the trainer device 120, the stress management system 140, or some combination thereof. The client device 110 can be a mobile computing device, such as a smartphone, a tablet, a headset (e.g., an augmented reality headset), etc. The client device 110 receives sensor data from the sensor assembly 105. The client device 110 may process the sensor data to determine one or more performance scores (e.g., stress regulation score).

The client device 110 may provide, via the network 130, stress performance data (e.g., sensor data, the one or more scores, etc.) to the trainer device 120 and/or the stress management system 140. Stress performance data is data associated with the stressor that describes performance of the user. Stress performance data may include, e.g., some or all of the sensor data, the one or more performance scores, video data (e.g., as captured by a camera assembly of the client device), or some combination thereof. In some embodiments, the client device 110 may pre-process some or all of the sensor data before providing it to the trainer device 120 and/or the stress management system 140.

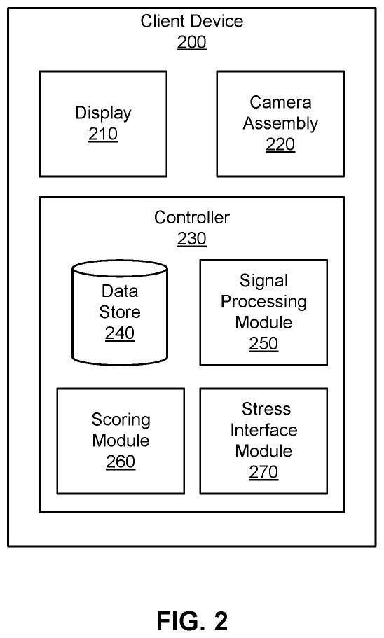

The client device 110 may present alerts (e.g., to the user). In some embodiments, client device 110 may generate an alert for the user based in part on the performance data of the user. In some embodiments, the client device 110 may receive alerts from, e.g., the trainer device 120 and/or the stress management system 140. An alert provides notice to the user about some aspect of their performance relating to a stressor. An alert may include, e.g., haptic feedback, an audio cue (e.g., alarm), a visual cue (e.g., flashing light), or some combination thereof. In some embodiments, the alert may include a course of action for the user to take. For example, the performance data may indicate that the user is too excited and the alert may include a course of action to help the user calm down (e.g., instructions for breathing exercises). The client device 110 is described in detail below with regard to FIG. 2.

The trainer device 120 may be used to evaluate performance of one or more users during stressor events. In some embodiments, the trainer device 120 can perform calculations to compare users to each other and analyze the performance of a team of users as a whole. The trainer device 120 is communicatively coupled to one or more client devices (e.g., the client device 110) via the network 130, and may also be communicatively coupled to the stress management system 140 via the network 130. The trainer device 120 may receive (e.g., via the network 130) stress performance data from the one or more client devices that are each associated with different users.

The trainer device 120 may analyze received stress performance data to generate performance information. Performance information is information that describes how one or more users of one or more client devices respond to stressors. Performance information may include, e.g., performance scores, sensor data, video data of stressors, etc. In some embodiments, the trainer device 120 monitors values of performance scores of users associated with client devices. The trainer device 120 may compare the values of the performance scores to corresponding threshold values. And if a score of a performance score of a user is below its corresponding threshold value, the trainer device 120 may generate an instruction to provide an alert to the client device associated with the user, and provide the generated alert to the client device.

The trainer device 120 presents the performance information to an operator (e.g., trainer) of the trainer device 120 via a training interface. The training interface is a graphical user interface that presents performance information and allows the operator to interact with the trainer device 120. The training interface may, e.g., receive trainer values from the operator to evaluate how well a user performed in one or more categories (e.g., communication, de-escalation, etc.) during a stressor (e.g., experienced over a time period). The trainer values are from the operator, and are not based on physiological data from the client device 110. For example, a trainer may rate how well a user performs in a category (e.g., communication) during the stressor using a scale of 1 to 10 (from worst performance to best performance). The trainer values and the performance data (e.g., the performance scores) may be provided to the stress management system 140 (e.g., to train a stress performance model to predict overall performance of a user of the client device).

The operator may configure the trainer device 120 to send alerts once certain conditions are satisfied. For example, the operator may configure the trainer device 120 to send an alert if a performance score falls below some threshold value. In some embodiments, the operator may instruct the trainer device 120 via the training interface to provide an alert to a user of a client device. In some embodiments, the training interface may also allow the operator to communicate directly with a user of a client device (e.g., the client device 110) via text messages, audio communications (e.g., phone call), video communications (e.g., video call), or some combination thereof.

The client device 110, the trainer device 120, and the stress management system 140 can communicate with each other via the network 130. The network 130 is a collection of computing devices that communicate via wired or wireless connections. The network 130 may include one or more local area networks (LANs) or one or more wide area networks (WANs). The network 130, as referred to herein, is an inclusive term that may refer to any or all of standard layers used to describe a physical or virtual network, such as the physical layer, the data link layer, the network layer, the transport layer, the session layer, the presentation layer, and the application layer. The network 130 may include physical media for communicating data from one computing device to another computing device, such as MPLS lines, fiber optic cables, cellular connections (e.g., 3G, 4G, or 5G spectra), or satellites. The network 130 also may use networking protocols, such as MQTT, TCP/IP, HTTP, SSH, SMS, FTP, to transmit data between computing devices. In some embodiments, the network 130 may include Bluetooth or near-field communication (NFC) technologies or protocols for local communications between computing devices. The network 130 may transmit encrypted or unencrypted data.

Note that in some cases the client device 110 and/or the trainer device 120 may not have connectivity to the internet. For example, military personnel may be in situations where there is no connectivity to the internet. As such, in some embodiments, the network 130 may be a local network between the client device 110 and the trainer device 120. This allows communication between the client device 110 and the trainer device 120 during and/or subsequent to a stressor. And once internet connectivity is re-established, the client device 110 and/or the trainer device 120 may also communicate with the stress management system 140.

The stress management system 140 evaluates user performance while experiencing a stressor. In the embodiment of FIG. 1, the stress management system 140 includes an evaluation module 150, a machine-learning training module 160, and a data store 170. Some embodiments of the stress management system 140 have different components than those described here. Similarly, in some cases, functions can be distributed among the components in a different manner than is described here.

The evaluation module 150 may receive stress performance data from one or more client devices and/or one or more trainer devices. For a given client device, the evaluation module 150 may apply the stress performance data to a stress performance model to predict overall performance of a user of the client device. In some embodiments, the stress performance model may generate one or more visualizations associated with the performance of the user. The one or more visualizations may include predicted performance scores for users of the client devices, predicated trainer values, etc. The stress performance model may be a machine learned model. The evaluation module 150 may perform an action based in part on stress performance data. For example, in some embodiments, the stress management system 140 may perform an action (e.g., send a client device an alert) based on received stress performance data.

The machine-learning training module 160 trains the stress performance model used by the stress management system 140. The stress performance model may be formed from one or more machine learning models. Example machine learning models include regression models, support vector machines, naïve bayes, k-means, decision trees, k nearest neighbors, random forest, boosting algorithms, and hierarchical clustering. The machine learning models may also include neural networks (e.g., perceptrons, multilayer perceptrons, convolutional neural networks, recurrent neural networks, sequence-to-sequence models, generative adversarial networks, or transformers).

The stress performance model may include a set of parameters. The set of parameters may be parameters that the stress performance model uses to process an input. For example, a set of parameters for a linear regression model may include weights that are applied to each input variable in the linear combination that comprises the linear regression model. Similarly, the set of parameters for a neural network may include weights and biases that are applied at each neuron in the neural network. The machine-learning training module 160 generates the set of parameters for a machine learning model by “training” the machine learning model. Once trained, the machine learning model uses the set of parameters to transform inputs into outputs.

The machine-learning training module 160 trains the stress performance model based on a set of training examples. Each training example includes input data to which the machine learning model is applied to generate an output. For example, each training example may include sensor data (e.g., GSR data, position data, etc.), one or more performance scores, trainer values, video data for stressors, stressor description data, time periods corresponding to occurrences of stressors, or some combination thereof. In some cases, the training examples may also include a label which represents an expected output of the machine learning model. In these cases, the stress performance model is trained by comparing its output from input data of a training example to the label for the training example.

The machine-learning training module 160 may apply an iterative process to train stress performance model whereby the machine-learning training module 160 trains the stress performance model on each of the set of training examples. To train the stress performance model based on a training example, the machine-learning training module 160 applies the stress performance model to the input data in the training example to generate an output. The machine-learning training module 160 scores the output from the stress performance model using a loss function. A loss function is a function that generates a score for the output of the stress performance model such that the score is higher when the stress performance model performs poorly and lower when the stress performance model performs well. In cases where the training example includes a label, the loss function is also based on the label for the training example. Some example loss functions include the mean square error function, the mean absolute error, hinge loss function, and the cross-entropy loss function. The machine-learning training module 160 updates the set of parameters for the machine learning model based on the score generated by the loss function. For example, the machine-learning training module 160 may apply gradient descent to update the set of parameters.

The data store 170 stores data used by the stress management system 140. For example, the data store 170 stores sensor data, performance scores, trainer values of performance scores, video data for stressors, stressor description data, time periods corresponding to occurrences stressors, alerts, visualizations, etc., for use by the stress management system 140. The data store 170 also stores trained machine learning models (e.g., the stress performance model) trained by the machine-learning training module 160. For example, the data store 170 may store the set of parameters for a trained machine learning model on one or more non-transitory, computer-readable media. The data store 170 uses computer-readable media to store data, and may use databases to organize the stored data.

FIG. 2 illustrates a block diagram of a client device 200, in accordance with one or more embodiments. The client device 200 is an embodiment of the client device 110. In the embodiment of FIG. 2, the client device 200 includes a display 210 and a controller 230, and optionally includes a camera assembly 220. Some embodiments of the client device 200 have different components than those described here. For example, the client device 200 may include some or all of the plurality of sensors of the sensor assembly 105. Similarly, in some cases, functions can be distributed among the components in a different manner than is described here.

The display 210 is configured to present information to a user of the client device 200. The display 210 may present, for example, an interface in accordance with instructions from the stress interface module 270. The training interface may provide, e.g., video data (e.g., captured during the stressor), sensor data, performance scores, etc. The display 210 may be a touch display such that the user may provide commands to the client device 200 via the display 210.

The camera assembly 220 captures video data of a local area of the client device 200. The camera assembly 220 includes one or more cameras and one or more microphones configured to capture video information of the local area. The camera assembly 220 is configured to capture video data in accordance with instructions from the controller 230. The camera assembly 220 may be integrated into the client device 200 (e.g., a camera that is part of a smart phone). In other embodiments, the camera assembly 220 is physically separate from the client device 200 but is communicatively coupled to the client device 200 (e.g., camera connected to the client device 200 via a BLUETOOTH connection).

The controller 230 controls operation of the client device 200. In the embodiment of FIG. 2, the controller 230 includes a data store 240, a signal processing module 250, a scoring module 260, and a stress interface module 270. Some embodiments of the controller 230 have different components than those described here. Similarly, functions can be distributed among the components in different manners than described here. For example, some functions of the controller may be performed external to the client device 200. The user may opt in to allow the controller 230 to transmit data captured by the client device 200 and/or a sensor assembly to systems external to the client device 200, and the user may select privacy settings controlling access to any such data.

The data store 240 stores data for use by the client device 200. Data in the data store 240 may include sensor data from the sensor assembly, performance scores, video data for stressors, time periods corresponding to occurrences stressors, alerts, courses of action, and other data relevant for use by the client device 200, or any combination thereof.

The signal processing module 250 may be configured to process the sensor data received from the sensor assembly. The received sensor data describes in part physiological data of the user collected over a time period. The time period includes a pre-target time period, a target time period, and a post-target time period. The sensor data includes GSR data, and may also include position data, PPG sensor data, etc. The signal processing module 250 is configured to determine a target period within the time period. A start time and an end time define a target period. In some embodiments the start time and end time are received from a trainer device (the trainer device 120). In some embodiments, the signal processing module 250 uses the position data (e.g., acceleration) and/or the PPG sensor data (e.g., heartrate) to determine the target time period. For example, the PPG sensor may indicate changes in heart rate of the user that indicate occurrence of a stressor. The target period has a start period and an end period that together define a range of times that include a ramp up time ahead of a stressor, a time period where the stressor occurs, and a ramp down time after occurrence of the stressor.

The signal processing module 250 may be configured to pre-process some or all of the sensor data (e.g., over some or all of the time period). Pre-processing may include, e.g., performing a quality check, cleaning, signal separation, some other processing to prepare sensor data for evaluation, or some combination thereof. The signal processing module 250 may provide pre-processed data to, e.g., the scoring module 260, a trainer device (e.g., the trainer device 120), a stress management system (e.g., the stress management system 140), or some combination thereof.

The signal processing module 250 may be configured to perform a quality check to determine whether the sensor data is likely to be valid. The signal processing module 250 may apply, e.g., the sensor data (e.g., the GSR data) to a model (e.g., machine learned model) to determine whether the data is valid or not. The machine learned model determines whether the sensor data satisfies one or more conditions. Note conditions may differ for different types of sensor data (e.g., GSR data v. PPG data). For GSR data, conditions may include, e.g., median amplitude of the GSR data less than a threshold value, number of sudden drops or rises in the GSR data greater than some threshold value, and the GSR data is not near zero for more than a threshold value of time unless at end of the GSR data. If the sensor data (e.g., the GSR data) fails the quality check, the signal processing module 250 can notify the user that the sensor data is potentially corrupted.

In some embodiments, the signal processing module 250 may be configured to clean some or all of the sensor data. For example, once the signal processing module 250 has validated the quality of the sensor data, the signal processing module 250 may clean the sensor data (e.g., GSR data). For GSR data, the signal processing module 250 may, e.g., perform median filtering, check for artifacts, perform filtering (e.g., spline interpolation), downsampling, etc. For other types of sensor data, other forms of processing may be used to clean the data.

The signal processing module 250 may be configured to process the cleaned GSR data to perform signal separation. Signal separation is a process through which the signal processing module 250 determines a tonic component associated with the GSR data, a phasic component associated with the GSR data, or both. The tonic component is a processed component of the GSR data that has a frequency below a low frequency threshold value, and the phasic component is a processed component of the GSR data that has a frequency above a threshold value that is at least the low frequency threshold value. In some embodiments, for one or more of the pre-target time period, the target period, and the post-target time period, the signal processing module 250 may determine, a tonic component of the GSR data and a phasic component of the GSR data.

The scoring module 260 may determine one or more performance scores based in part on the tonic component associated with the GSR data and the phasic component associated with the GSR data. A performance score quantifies an aspect of performance of the user in response to experiencing a stressor. The one or more performance scores may include, e.g., a stress regulation score, an anxiety score, a vigilance score, a recovery score, some other score that quantifies a performance of a user, or some combination thereof.

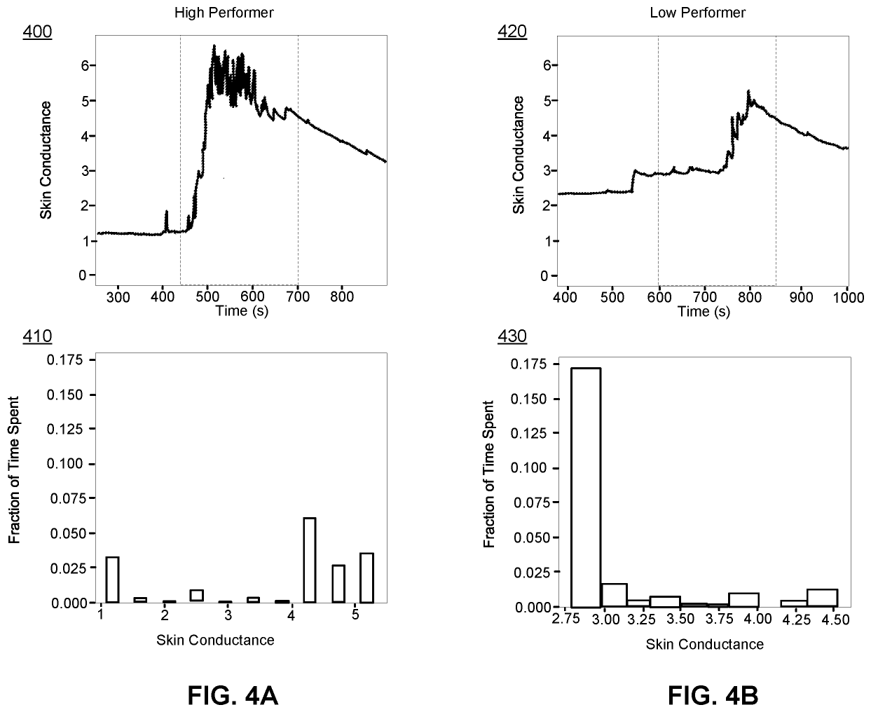

The scoring module 260 may be configured to generate a representation of the tonic component of the GSR data that is indicative of how the user's physiological state changes during a high stress event. In one example implementation, the scoring module 260 derives a histogram using the tonic component of the GSR data. Here, the scoring module 260, may filter (e.g., low pass filter) the tonic component of the GSR data. The scoring module 260 bins the tonic component (e.g., that may be pre-processed) of GSR data into a range of values, such that the horizontal axis corresponds to a range of skin conductance. The scoring module 260 may convert the vertical axis from number of counts per bin to fraction of time (over the target time period) spent in a bin to generate the histogram. Some example histograms are shown and described below with regard to, e.g., FIGS. 4A and 4B. In other example implementations, the scoring module 260 may derive a best fit line or other representation of the tonic component of the GSR data indicative of changes in state.

The scoring module 260 may be configured to determine a stress regulation score using the histogram or other representation. The stress regulation score describes a capability of the user to regulate stress during the target period. At a high level the stress regulation score is a comparison of time spent with a high skin conductance relative to time spent at a low skin conductance. As such, a user who regulates stress well during the target period tends to have skin conductance values at high levels for longer periods of time than a user who does not regulate stress well during a target period. An example embodiment of a stress regulation score using a histogram-based representation is calculated as follows. The scoring module 260 may divide the histogram into two portions. In most cases there is a peak (e.g., local maximum) on a left side of the histogram (as these are the lowest values representing the baseline stress before the user starts rising their level). However, for high performers there is generally is a peak (e.g., local maximum) on a right side of the histogram as well at the high values (representing the user's ability to maintain a high value rather than constantly raising their stress level without maintaining it). By dividing the histogram into two portions, the scoring module 260 is able to captures both of these values. For example, in some embodiments, the scoring module 260 divides the histogram into a first portion (e.g., lower skin conductance values) and a second portion (e.g., higher skin conductance values) that are separated at a division point. In some embodiments, the division point is a center point of the horizontal axis of the histogram. The scoring module 260 determines a characteristic value for the first portion and a characteristic value for the second portion. In some embodiments, the scoring module 260 determines that a peak value (vertical axis) of a portion of a histogram is a characteristic value for that portion. In some embodiments, the scoring module 260 may average the values of a portion of the histogram to determine the characteristic value for that portion. The scoring module 260 determines a ratio of the second characteristic value to the first characteristic value. The value of the ratio is the stress regulation score during the target period for the user.

The scoring module 260 may be configured to determine an anxiety score using the tonic component. The anxiety score is a measure of anxiety of the user prior to the target period (e.g., during the pre-target time period). Increasing anxiety generally manifests as an increase in slope of the pre-processed tonic component of the GSR data. The scoring module 260 may, e.g., compute a slope value of the pre-processed tonic component of the GSR data. In some embodiments, the scoring module 260 generates the anxiety score from the computed slope value over a range of time values in the pre-target time period (e.g., from 1 minute prior to a beginning of the target period). For example, the scoring module 260 may use the computed slope as the anxiety score.

The scoring module 260 may be configured to determine a recovery score using the tonic component. The recovery score is a measure of how fast the user recovers from a stressor. In general, recovery manifests as a negative rate of change of the pre-processed tonic component of the GSR data. In some embodiments, the scoring module 260 computes a slope value over a range of time values in the post-target time period (e.g., from the end of the target period to 1 minute after the target period). The scoring module 260 may generate the recovery score from the computed slope value. The scoring module 260 may, e.g., using the computed slope value as the recovery score.

The scoring module 260 may be configured to determine a vigilance score using the phasic component. The vigilance score is a measure of vigilance of the user during the target period. The scoring module 260 determines peak locations within the phasic component of the GSR data. Vigilance generally manifests as a combination of tonic and phasic features. The scoring module 260 may determine the vigilance score by, e.g., determining a slope for the tonic component of the target period, and determining a frequency of peaks over the target period. The scoring module 260 may weight (e.g., 0.5) the slope and separately weight (e.g., 0.5) the determined frequency, and sum the weighted values to generate a vigilance score. In some embodiments, values of the weights may be adjusted as part of or after training the stress performance model.

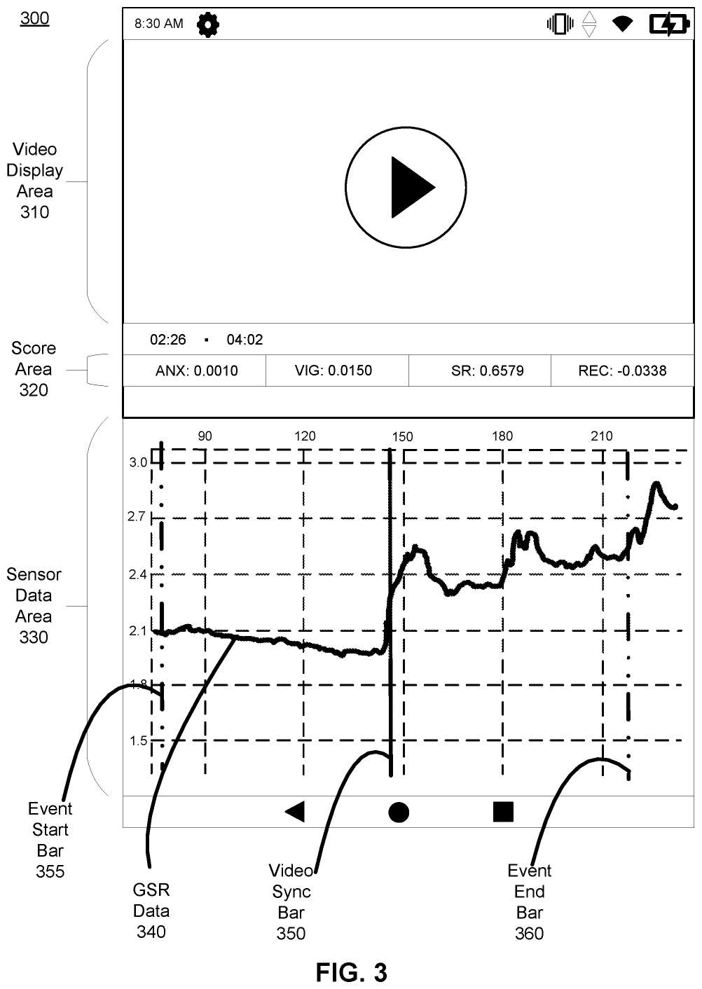

The stress interface module 270 may be configured to generate an interface to present stress performance data to the user. The interface is a stress performance graphical user interface that presents performance data. Stress performance data is data associated with the stressor that describes performance of the user. Stress performance data may include, e.g., some or all of the sensor data (e.g., GSR data), the one or more performance scores, video data (e.g., captured by the camera assembly 220), or some combination thereof. An example in stress performance graphical user interface is shown and described below with regard to FIG. 3. The stress interface module 270 may be configured to present the interface using the display 210. In some embodiments, the stress interface module 270 may highlight the presented stress performance scores based in part on their respective values.

In some embodiments, the scoring module 260 may compare some or all of the scores (e.g., stress regulation score, the anxiety score, the recovery score, the vigilance score) for the user to corresponding scores of other users. For a given score (e.g., stress regulation score) of a user, the scoring module 260 ranks the score relative to corresponding scores (e.g., stress regulation scores) of other users. The score of the user may be color coded based on its ranking (e.g., red if bottom 25%, green if top 25%, and orange otherwise). Some or all of the color-coded scores of the user may be presented via the interface (e.g., as described below with regard to FIG. 3). In some embodiments, the stress interface module 270 may provide, via the network 130, stress performance data (e.g., sensor data, the one or more scores, etc.) to the trainer device 120 and/or the stress management system 140. In some embodiments, the stress interface module 270 may pre-process some or all of the sensor data before providing it to the trainer device 120 and/or the stress management system 140.

The stress interface module 270 may instruct the client device 200 to present alerts (e.g., to the user). An alert provides notice to the user about some aspect of their performance relating to a stressor. In some embodiments, stress interface module 270 may generate an alert for the user based in part on the performance data of the user. In some embodiments, the client device 110 may receive alerts from, e.g., a trainer device (e.g., the trainer device 120) and/or a stress management system (e.g., the stress management system 140).

In some embodiments, the alert may include a course of action for the user. The stress interface module 270 may be configured to select a course of action, from a plurality of different courses of action, to improve stress performance of the user based in part on the performance data (e.g., the one or more performance scores). The plurality of courses of action each address specific aspects of stress performance (e.g., some may work to improve the anxiety score, some may work to improve stress regulation scores, etc.). Courses of action may include, e.g., brief exercises to integrate into training such as breathwork, mental imagery, or longer term exercise plans such as daily meditative work-outs, educational videos, some other course of action that may positively impact stress performance, or some combination thereof. Once selected, the stress interface module 270 may prompt the user to perform the selected course of action. For example, the performance data may indicate that the user is too excited and the alert may include a course of action to help the user calm down (e.g., instructions for breathing exercises). Note in some embodiments the user may provide feedback after an exercise so that the controller 230 may learn which exercises work best for the user.

Note in some embodiments, the alerts and/or courses of action are provided after the target period. In these cases, the alerts and/or course of action are useful in providing feedback that the user can use to improve stress performance when subject to stressors in the future. In other embodiments, the alerts and/or courses of action are provided in real time. The real time alerts and/or courses of action may be determined by the client device 200, the trainer device, the stress management system, or some combination thereof. This may be particularly helpful in cases where the performance data of the user shows that the user is not responding to the stressor well (e.g., anxiety score is showing the user as being in a state of debilitating anxiety). For example, in some embodiments, the stress interface module 270 can provide a real time alert and/or course of action to the user that instructs the user to disengage from the stressor.

FIG. 3 is an example stress performance graphical user interface (GUI) 300, in accordance with one or more embodiments. The stress performance GUI 300 may be presented on a client device (e.g., the client device 110). The stress performance GUI 300 is a user interface that provides information about user performance relating to a stressor experienced by the user. In the illustrated embodiment, the stress performance GUI 300 includes a video display area 310, a score area 320, and a sensor data area 330. In other embodiments, the stress performance GUI 300 includes different or additional elements. In addition, the functions may be distributed among the elements in a different manner than described.

The video display area 310 presents video data associated with the stressor. The video data may have been captured, e.g., using a camera assembly (e.g., the camera assembly 220) of the client device during the target period. The video display area 310 may include one or more soft buttons to enable the user to interact (e.g., play, stop, fast forward, rewind, etc.) with the video data.

The score area 320 presents one or more of the performance scores of the user. For example, in FIG. 3 an anxiety score (ANX) of 0.0010 is shown, a vigilance score (VIL) of 0.0150 is shown, a stress regulation score (SR) of 0.6579 is shown, and a recovery score (REC) of −0.0338 is shown. Note in other embodiments, the values of one or more of the presented scores may be rescaled to some other form (e.g., 1 to 100%). For example, instead of presenting a vigilance score of 0.0150, the score area 320 may rescale this to percentage form and show 80% (i.e., the vigilance score was in the top 20%). In the illustrated embodiment the scores are based on various parts of the time period (e.g., anxiety score is based on data from pre-target period, whereas the stress regulation score is associated with the target period). In alternate embodiments, one or more of the scores may update in real-time. In some embodiments, the score area 320 may color code scores based on their values. For example, if a performance score is below a first threshold (e.g., lowest 25% of users), the score area 320 may present the score (e.g., highlight the box containing the performance score) in red; if the performance score is between the first threshold and a second threshold (e.g., top 25% of users), the score area 320 may present the score in yellow; and if the performance score is above the second threshold, the score area 320 may present the score in green.

The sensor data area 330 presents some or all of the sensor data. For example, as shown in FIG. 3, the sensor data area 330 is presenting GSR data 340 over a portion of the target period (e.g., from ˜65 to ˜230 seconds). The sensor data area 330 includes a video sync bar 350. The video sync bar 350 is synchronized to the video data being presented in the video display area 310. As such, as the video data plays the video sync bar 350 advances in time along the GSR data 340. In some embodiments, the user may move the video sync bar 350 to a particular time value along the GSR data 340 and the video display area 310 automatically adjusts the playback of the video data such that it corresponds to the particular time value. In some embodiments, the sensor data area 330 includes an event start bar 355, and an event end bar 360. The event start bar 355 illustrates where the event begins relative to the GSR data 340 (e.g., a start time of the target period), and the event end bar 360 illustrates where the event ends relative to the GSR data 340 (e.g., an end time of the target time period). The client device places the event start bar 355 and the event end bar 360 based on the determined start time and end time of the target time period (e.g., received from training device, or determined from PPG data, position data, etc.).

FIG. 4A is a chart 400 showing skin response and an associated histogram 410 for a user who regulates stress well during a target period, in accordance with one or more embodiments. A user who regulates stress well during a target period may be referred to as a high performer (e.g., stress regulation score that puts the user in the top 25% of users). Likewise, a user who does not regulate stress well during a target period may be referred to as a low performer (e.g., stress regulation score that puts the user in the bottom 25% of all users).

The chart 400 shows GSR data describing skin conductance (u-Siemens) as a function of time (seconds). In the example of FIG. 4A, the presented GSR data spans a time period of 200-900 seconds. The dashed lines in the chart 400 represent a target period of time within the time period. The target period is a range of times that includes a ramp up time ahead of a stressor, a time period where the stressor occurs, and a ramp down time after occurrence of the stressor. The GSR data from the target time period may be used to generate a histogram (e.g., the histogram 410) that can be used to determine a stress regulation score. The histogram may be generated by, e.g., a scoring module (e.g., the scoring module 260) of a client device, a trainer device (e.g., the trainer device 120), a stress management system (e.g., the stress management system 140), or some combination thereof.

Using the histogram 410 a stress regulation score may be calculated. For example, the histogram 410 may be divided into a first portion and a second portion (e.g., at mid-point). For example, the first portion may be for skin conductance values less than 3 and the second portion may be for skin conductance values greater than 3. A peak value of the second portion is ˜0.060 and a peak value of the first portion is ˜0.03. In this embodiment, the ratio between these values provides us with a stress regulation score of 2.0. As 2.0 corresponds to a value that is in the top 25% of users, and indicates the user is a “high performer.”

FIG. 4B is a chart 420 showing skin response and an associated histogram 430 for a user who does not regulate stress well during a target period, in accordance with one or more embodiments. The chart 420 shows GSR data describing skin conductance (u-Siemens) as a function of time (seconds). In the example of FIG. 4B, the presented GSR data spans a time period of 380-1000 seconds. The dashed lines in the chart 420 represent a target period of time within the time period. Note that the GSR data in the chart 420 in general has much lower skin conductance as a function of time than the GSR data in the chart 400. The GSR data from the target time period may be used to generate the histogram 430. The histogram 430 can be used to determine a stress regulation score using a process substantially similar to that described above for FIG. 4A. Using this process, a stress regulation score of 0.074 (=˜0.0125/0.017) for the user is determined. As 0.74 corresponds to a value that is in the bottom 25% of users, is below a and indicates the user is a “low performer.”

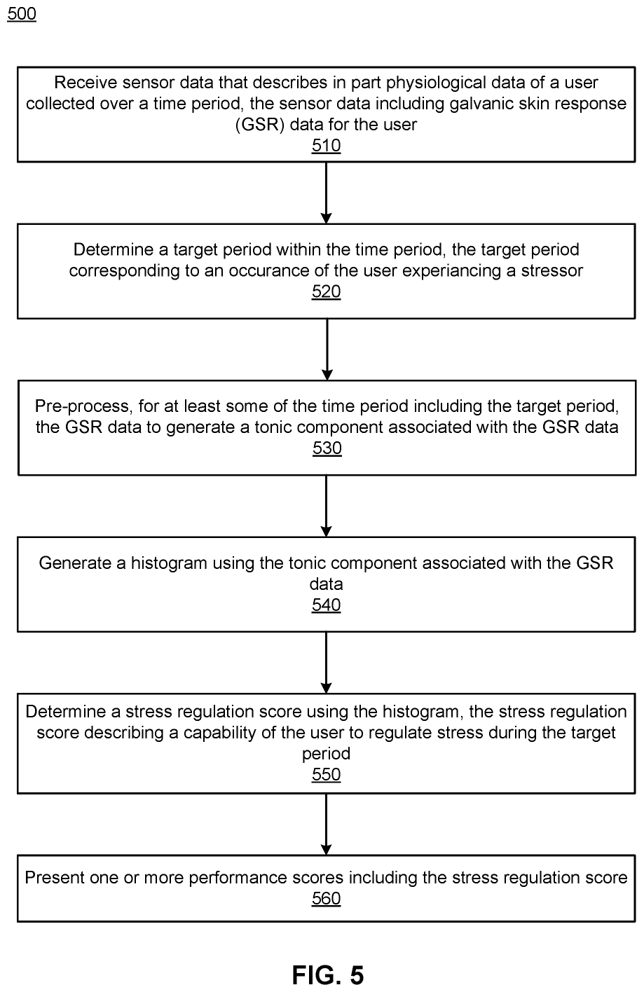

FIG. 5 is a flowchart illustrating a process 500 for evaluating stress performance on a client device, in accordance with one or more embodiments. The process 500 shown in FIG. 5 may be performed by components of a client device (e.g., the client device 110). Other entities may perform some or all of the steps in FIG. 5 in other embodiments. Embodiments may include different and/or additional steps, or perform the steps in different orders.

The client device receives 510 sensor data that describes in part physiological data of a user collected over a time period. The received sensor data includes GSR data for the user. The client device may receive the sensor data from, e.g., a sensor assembly (e.g., the sensor assembly 105). In some embodiments, the client device may also receive sensor data from sensors (e.g., position sensor) that are part of the client device.

The client device determines 520 a target period within the time period. The target period corresponds to an occurrence of the user experiencing a stressor. The target period is a range of times that includes a ramp up time ahead of a stressor, a time period where the stressor occurs, and a ramp down time after occurrence of the stressor. In some embodiments, the client device determines the target period (e.g., a start period to an end period) using the sensor data (e.g., PPG data and/or position data). In other embodiments, the client device may determine the target period using data from the user, a trainer device (e.g., the trainer device 120), a stress management system (e.g., the stress management system 140), or some combination thereof.

The client device pre-processes 530, for at least some of the time period including the the target period, the GSR data to generate a tonic component associated with the GSR data. For example, the client device may pre-process the GSR data for the target time period, and may also pre-process the GSR data for the pre-target time period and/or the post-target time period. In some embodiments, the client device may also pre-process, for the time period, the GSR data to generate a phasic component associated with the GSR data. Pre-processing may include performing, via a signal processing module (e.g., the signal processing module 250) a quality check of the GSR data in order to validate the GSR data. If the GSR data fails the quality check, the client device can notify the user that the data is potentially corrupted. The signal processing module cleans (e.g., perform median filtering, check for artifacts, perform filtering (e.g., spline interpolation), downsampling, etc.) the GSR data to form cleaned GSR data. The signal processing module processes the cleaned GSR data to perform signal separation to determine the tonic component from the cleaned GSR data, and in some embodiments the phasic component from the cleaned GSR data.

The client device generates 540 a histogram using the tonic component associated with the GSR data. A scoring module (e.g., the scoring module 260) of the client device may filter (e.g., low pass filter) the tonic component of the GSR data. The scoring module bins the filtered tonic component of GSR data into a range of values, such that the horizontal axis corresponds to a range of skin conductance. The scoring module converts the vertical axis from number of counts per bin to fraction of time (over the target time period) spent in a bin to generate the histogram.

The client device determines 550 a stress regulation score using the histogram. The stress regulation score describes a capability of the user to regulate stress during the target period. The scoring module may divide the histogram into a first portion and a second portion that are separated at a division point. In some embodiments, the division point is a center point of the horizontal axis of the histogram. The scoring module determines a characteristic value for the first portion and a characteristic value for the second portion. In some embodiments, the scoring module determines that a peak value (vertical axis) of the first portion of the histogram is a characteristic value for the first portion, and determines that a peak value (vertical axis) of the second portion of the histogram is a characteristic value for the second portion. In some embodiments, the scoring module may average the values of the first portion of the histogram to determine the characteristic value for the first portion, and average the values of the second portion of the histogram to determine the characteristic value of the second portion. The scoring module determines a ratio of the second characteristic value to the first characteristic value. The value of the ratio is the stress regulation score during the target period for the user.

In alternative implementations, a different representation of the tonic component of the GSR data may be derived that does not necessarily include a histogram. In such embodiments, steps 540, 550 may be modified to generate a different representation of the tonic component of the GSR data and determine a stress regulation store based on the representation. For example, a best fit line of the tonic component may be derived and the scoring module may apply a function to the best fit line that characterize how the slope of the line changes over time, which may be indicative of the user's stress regulation ability. Alternatively, other representations may be employed to derive a similar assessment.

The client device presents 560 one or more performance scores including the stress regulation score. The client device may present stress performance data that includes the one or more performance scores with a stress performance GUI (e.g., the stress performance GUI 300) and a display (e.g., the display 210).

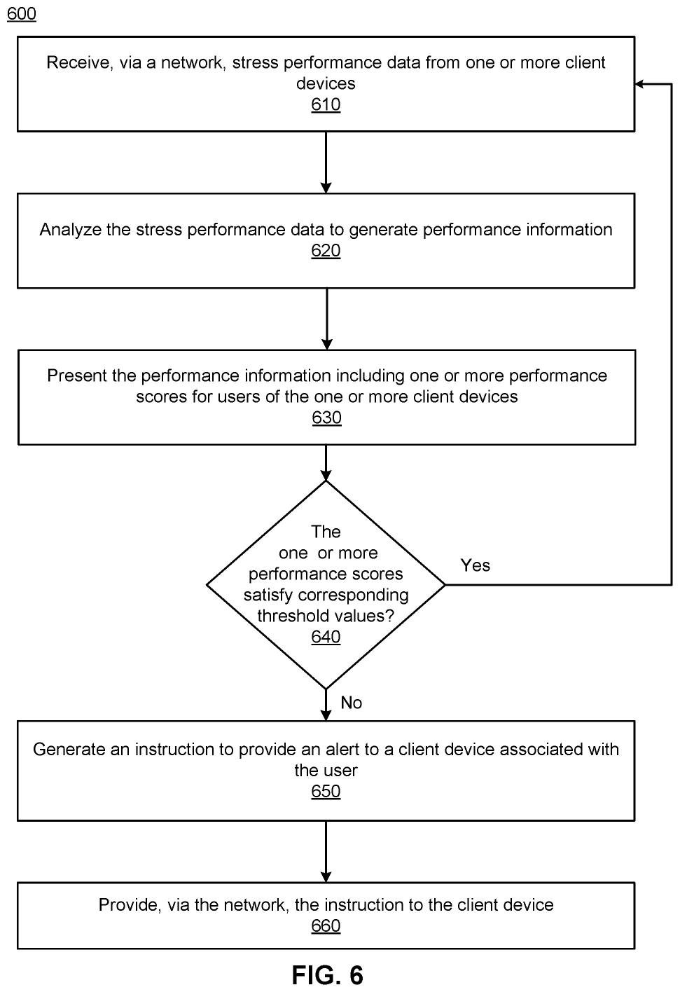

FIG. 6 is a flowchart illustrating a process for evaluating stress performance of multiple users on a trainer device, in accordance with one or more embodiments. The process 600 shown in FIG. 6 may be performed by components of a trainer device (e.g., the trainer device 120). Other entities may perform some or all of the steps in FIG. 6 in other embodiments. Embodiments may include different and/or additional steps, or perform the steps in different orders.

The trainer device receives 610 stress performance data from one or more client devices (e.g., client device 110). The trainer device receives the stress performance via a network. The network may be a local network.

The trainer device analyzes 620 the stress performance data to generate performance information. The performance information describes how one or more users of the one or more client devices respond to stressors. Performance information may include, e.g., performance scores, sensor data, video data of stressors, etc.

The trainer device presents 630 the performance information including one or more performance scores for users of the one or more client devices. The trainer device presents the performance information to an operator (e.g., trainer) of the trainer device via a training interface.

The trainer device determines 640 whether the one or more performance scores satisfy corresponding threshold values. The trainer device monitors values of performance scores of users associated with the one or more client devices. In some embodiments, the trainer device compares the values of the performance scores to corresponding threshold values. If the one or more performance scores satisfy corresponding threshold values, the process moves to step 610.

And if a score of a performance score of a user is below its corresponding threshold value, the trainer device generates 650 an instruction to provide an alert to a client device (of the one or more client devices) associated with the user. In some embodiments, the trainer device may determine a recommended course of action based on the performance data associated with the user (e.g., which of the one or more performance scores did not satisfy their corresponding threshold values). For example, the performance data may indicate that the user is too excited and the trainer may select a recommended course of action to help the user calm down (e.g., instructions for breathing exercises). In some embodiments, the trainer device may prompt an operator of the trainer device to provide a recommended course of action.

The trainer device provides 660 the instruction to the client device. The trainer device provides the alert via the network. Responsive to receiving the instruction, the client device provides the alert to the user.

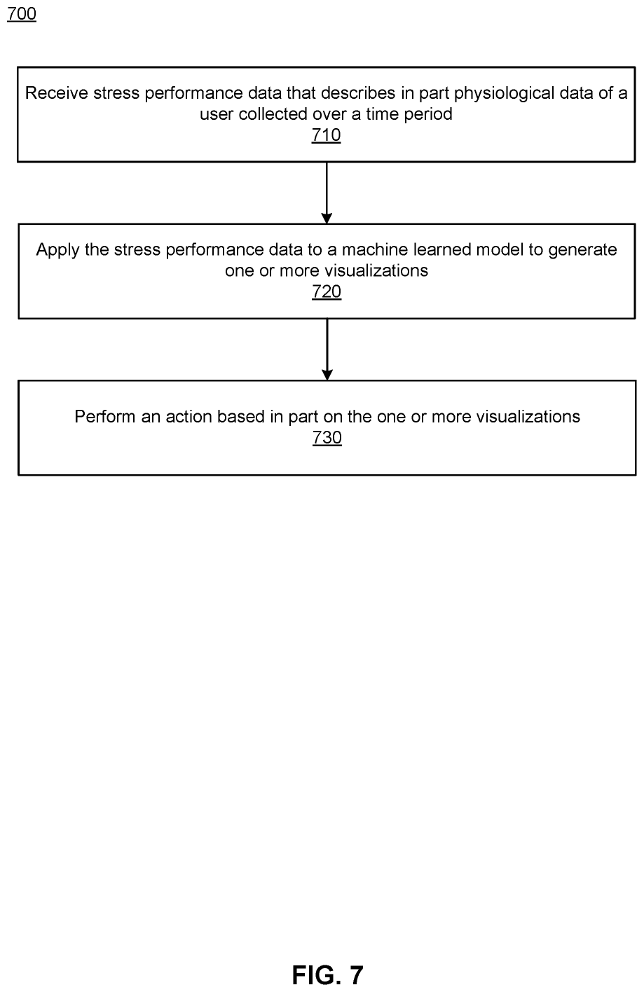

FIG. 7 is a flowchart illustrating a process for evaluating stress performance of multiple users using a machine learned model, in accordance with one or more embodiments. The process 700 shown in FIG. 7 may be performed by components of a stress management system (e.g., the stress management system 140). Other entities may perform some or all of the steps in FIG. 7 in other embodiments. Embodiments may include different and/or additional steps, or perform the steps in different orders.

The stress management system receives 710 stress performance data that describes in part physiological data of a user collected over a time period. The stress management system may receive the stress performance data (e.g., sensor data, performance scores, video data, etc.) from client devices (e.g., the client device 110) and/or trainer devices (e.g., the trainer device 120).

The stress management system applies 720 the stress performance data to a machine learned model to generate one or more visualizations.

The stress management system performs 730 an action based in part on the one or more visualizations.

### Additional Configuration Information

The foregoing description of the embodiments has been presented for illustration; it is not intended to be exhaustive or to limit the patent rights to the precise forms disclosed. Persons skilled in the relevant art can appreciate that many modifications and variations are possible considering the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
receiving sensor data that describes in part physiological data of a user collected over a time period, the sensor data including galvanic skin response (GSR) data for the user;
determining a target period within the time period, the target period corresponding to an occurrence of the user experiencing a stressor;
pre-processing, for at least some of the time period including the target period, the GSR data to determine a tonic component associated with the GSR data;
generating a histogram using the tonic component of the GSR data;
determining a stress regulation score using the histogram, the stress regulation score describing a capability of the user to regulate stress during the target period; and
presenting, via a display, one or more performance scores including the stress regulation score.

2. The method of claim 1, wherein determining the stress regulation score using the histogram, further comprises:
dividing the histogram into a first portion and a second portion;
determining a characteristic value for the first portion and a characteristic value for the second portion; and
determining a ratio of the characteristic value of the second portion to the characteristic value of the first portion, wherein the ratio is the stress regulation score.

3. The method of claim 1, wherein pre-processing, for at least some of the time period including the target period, the GSR data to determine the tonic component associated with the GSR data further comprises applying a low pass filter to the tonic component of the GSR data to generate a processed data set, the method further comprising:
determining slope values of curves within the processed data set; and
determining an anxiety score and a recovery score of the user for the target period based in part on the slope values,
wherein the anxiety score and the recovery score are two of the one or more performance scores.

4. The method of claim 1, further comprising:
pre-processing, for the target period, the GSR data to generate a phasic component associated with the GSR data;
determining peaks in the phasic component of the GSR data; and
determining a vigilance score of the user for the target period, the vigilance score based on the determined peaks,
wherein the vigilance score is one of the one or more performance scores.

5. The method of claim 1, wherein determining the target period within the time period, further comprises:
using the sensor data to determine a start period and an end period for the target period, wherein the sensor data used includes data from at least one of an inertial measurement unit and a photoplethysmogram sensor.

6. The method of claim 1, further comprising:
determining course of action, from a plurality of courses of action, to improve stress performance of the user based in part on the one or more performance scores; and
prompting the user to perform the action.

7. The method of claim 1, further comprising:
providing, via a network, the sensor data and the one or more performance scores to a stress management system that includes a machine learning model, wherein the machine learning model is trained in part using the one or more performance scores and the sensor data to predict performance scores for users based in part on GSR data for the users.

8. The method of claim 1, further comprising:
providing, via a network, the one or more performance scores to a trainer device,
wherein the trainer device:
analyzes the one or more performance scores and other performance scores associated with other users from other client devices to generate performance information of the user and the other users, and
presents the performance information to an operator of the trainer device.

9. The method of claim 8, wherein the trainer device determines that a value of at least one of the one or more performance scores is below a threshold value, and generates an instruction to provide an alert, the method further comprising:
receiving, via the network, the instruction from the trainer device; and
providing the alert to the user.

10. The method of claim 1, the method further comprising:
receiving, from a camera, video data of a local area of the user, the video data spanning at least the target period; and
presenting, via the display, the video data with the one or more performance scores.

11. A non-transitory computer-readable storage medium comprising stored instructions, the instructions when executed by a processor of a device, cause the device to:
receive sensor data that describes in part physiological data of a user collected over a time period, the sensor data including galvanic skin response (GSR) data for the user;
determine a target period within the time period, the target period corresponding to an occurrence of the user experiencing a stressor;
pre-process, for at least some of the time period including the target period, the GSR data to determine a tonic component associated with the GSR data;
generate a histogram using the tonic component of the GSR data;
determine a stress regulation score using the histogram, the stress regulation score describing a capability of the user to regulate stress during the target period; and
present, via a display, one or more performance scores including the stress regulation score.

12. The non-transitory computer-readable storage medium of claim 11, where the stored instructions to determine the stress regulation score using the histogram further comprises stored instruction that when executed cause the device to:
divide the histogram into a first portion and a second portion;
determine a characteristic value for the first portion and a characteristic value for the second portion; and
determine a ratio of the characteristic value of the second portion to the characteristic value of the first portion, wherein the ratio is the stress regulation score.

13. The non-transitory computer-readable storage medium of claim 11, further comprising stored instructions that when executed cause the device to:
apply a low pass filter to the tonic component of the GSR data to generate a processed data set;
determine slope values of curves within the processed data set; and
determine an anxiety score and a recovery score of the user for the target period based in part on the slope values,
wherein the anxiety score and the recovery score are two of the one or more performance scores.

14. The non-transitory computer-readable storage medium of claim 11, further comprising stored instructions that when executed cause the device to:
pre-process, for at least some of the time period including the target period, the GSR data to generate a phasic component associated with the GSR data;
determine peaks in the phasic component of the GSR data; and
determine a vigilance score of the user for the target period, the vigilance score based on the determined peaks,
wherein the vigilance score is one of the one or more performance scores.

15. The non-transitory computer-readable storage medium of claim 11, where the stored instructions to determine the target period within the time period further comprises stored instruction that when executed cause the device to:
use the sensor data to determine a start period and an end period for the target period, wherein the sensor data used includes data from at least one of an inertial measurement unit and a photoplethysmogram sensor.

16. The non-transitory computer-readable storage medium of claim 11, further comprising stored instructions that when executed cause the device to:
determine course of action, from a plurality of courses of action, to improve stress performance of the user based in part on the one or more performance scores; and
prompt the user to perform the action.

17. The non-transitory computer-readable storage medium of claim 11, further comprising stored instructions that when executed cause the device to:
provide, via a network, the sensor data and the one or more performance scores to a stress management system that includes a machine learning model, wherein the machine learning model is trained in part using the one or more performance scores and the sensor data to predict performance scores for users based in part on GSR data for the users.

18. The non-transitory computer-readable storage medium of claim 11, further comprising stored instructions that when executed cause the device to:
provide, via a network, the one or more performance scores to a trainer device,
wherein the trainer device is configured to:
analyze the one or more performance scores and other performance scores associated with other users from other client devices to generate performance information of the user and the other users, and
present the performance information to an operator of the trainer device.

19. The non-transitory computer-readable storage medium of claim 11, further comprising stored instructions that when executed cause the device to:
receive, from a camera, video data of a local area of the user, the video data spanning at least the target period; and
present, via the display, the video data with the one or more performance scores.

20. A system comprising:
a sensor assembly configured to collect sensor data that describes in part physiological data of a user over a time period, the sensor data including galvanic skin response (GSR) data for the user; and
a client device communicatively coupled to the sensor assembly, the client device including:
a display, and
a controller configured to:
determine a target period within the time period, the target period corresponding to an occurrence of the user experiencing a stressor,
pre-process, for at least some of the time period including the target period, the GSR data to determine a tonic component associated with the GSR data,
generate a histogram using the tonic component of the GSR data,
determine a stress regulation score using the histogram, the stress regulation score describing a capability of the user to regulate stress during the target period, and
instruct the display to present one or more performance scores including the stress regulation score.
